(19) United States
(12) Patent Application Publication
(10) Pub. No.: US 2019/0387879 A1
(43) Pub. Date: Dec. 26, 2019

(54) Emergency Response Kiosk

(71) Applicants: Jason J. Innella, Williamstown, NJ (US); Sean P. McKinney, Glassboro, NJ (US)

(72) Inventors: Jason J. Innella, Williamstown, NJ (US); Sean P. McKinney, Glassboro, NJ (US)

(21) Appl. No.: 16/452,408

(22) Filed: Jun. 25, 2019

Related U.S. Application Data
(60) Provisional application No. 62/689,536, filed on Jun. 25, 2018.

(51) Int. Cl. A47B 81/00

(52) U.S. Cl. CPC A47B 81/00; A61F 17/00

(57) ABSTRACT

An emergency response kiosk that stores vital emergency response supplies and functions as an on-site command and control center. The kiosk includes a tamper-proof locker box mounted atop a pedestal base. The locker box stores essential communication equipment, as well as other administrative gear and supplies necessary to establish on-site command and control operations. A hinged locker door and latch mechanism allows access only to authorized personnel. The kiosk is designed to be positioned at various strategic locations to provide first responders pre-staged decentralized access to vital medical supplies and equipment, as well as certain tactical gear and equipment in the event of a mass casualty event.

This application claims the benefit of U.S. Provisional Application No. 62/689,536 filed Jun. 25, 2018, the disclosure of which is hereby incorporated by reference.

This invention relates to a fixture for storing medical and tactical supplies and equipment for use by first responders and the public during emergencies, and in particular, a fixture that includes command and control equipment to organize and facilitate response to mass casualty events.

### BACKGROUND AND SUMMARY OF THE INVENTION

Any effective mitigation and response to mass casualty emergencies or critical incidents depends on the immediate coordination and management of first responders and security personnel, as well as the ready access, distribution and deployment of critical medical supplies and equipment at the scene of the emergency. Typically, first responders only have the resources available that they carry or transport to the location of the emergency. In mass casualty events and critical incidents, such as disasters and active shooter events, medical supplies and equipment, such as tourniquets, hemostatic agents and wound dressings, are needed in large quantities to treat large numbers of wounded. Retrieving additional medical supplies and equipment from centralized storage areas wastes critical time at the cost of lives. Moreover, the ability to facilitate on-site, real-time command and control communications and operations to coordinate and manage first responders, security, citizenry is critical.

The emergency response kiosk of this invention is a secure, stand-alone fixture that stores vital emergency response supplies and functions as an on-site command and control center. The kiosk includes a tamper-proof locker box mounted atop a pedestal base. The locker box stores essential communication equipment, as well as other administrative gear and supplies necessary to establish on-site command and control operations. A hinged locker door and latch mechanism allows access only to authorized personnel. The kiosk is designed to be positioned at various strategic locations to provide first responders pre-staged decentralized access to vital medical supplies and equipment, as well as certain tactical gear and equipment in the event of a mass casualty event or critical incident. Decentralizing vital medical supplies, communication equipment and tactical gear in secure stand alone kiosks, which can be strategically positioned in populated, high threat locations, mitigates the loss of life and improves the coordinated response to mass casualty and critical incidents.

The above described features and advantages, as well as others, will become more readily apparent to those of ordinary skill in the art by reference to the following detailed description and accompanying drawings.

### BRIEF DESCRIPTION OF THE DRAWINGS

The present invention may take form in various system and method components and arrangement of system and method components. The drawings are only for purposes of illustrating exemplary embodiments and are not to be construed as limiting the invention. The drawings illustrate the present invention, in which:

FIG. 1 is a perspective view of an exemplary embodiment of the emergency response kiosk of this invention shown in the closed position;

FIG. 2 is a perspective view of the kiosk of FIG. 1 shown in the open position; and

FIG. 3 is a front view of the kiosk of FIG. 1 shown in the open position.

### DESCRIPTION OF THE PREFERRED EMBODIMENT

In the following detailed description of the preferred embodiments, reference is made to the accompanying drawings that form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical, structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

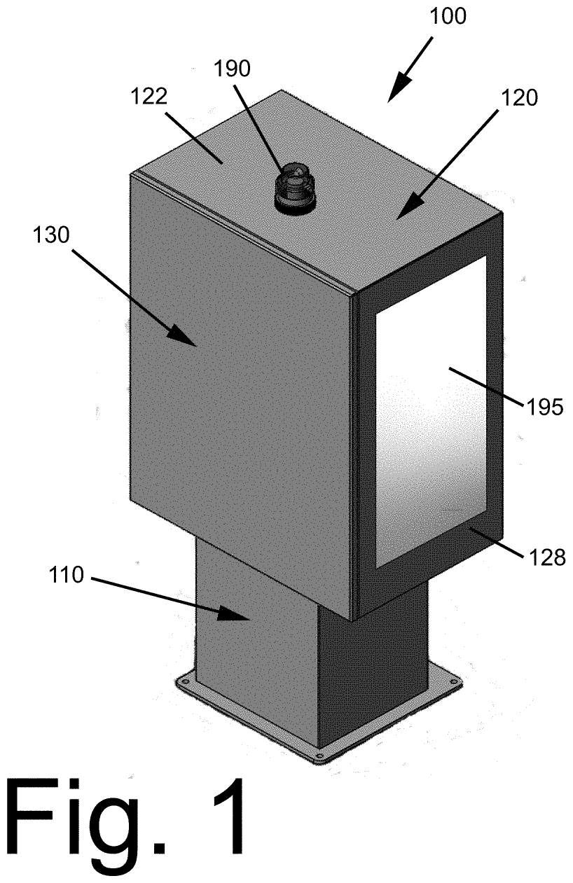

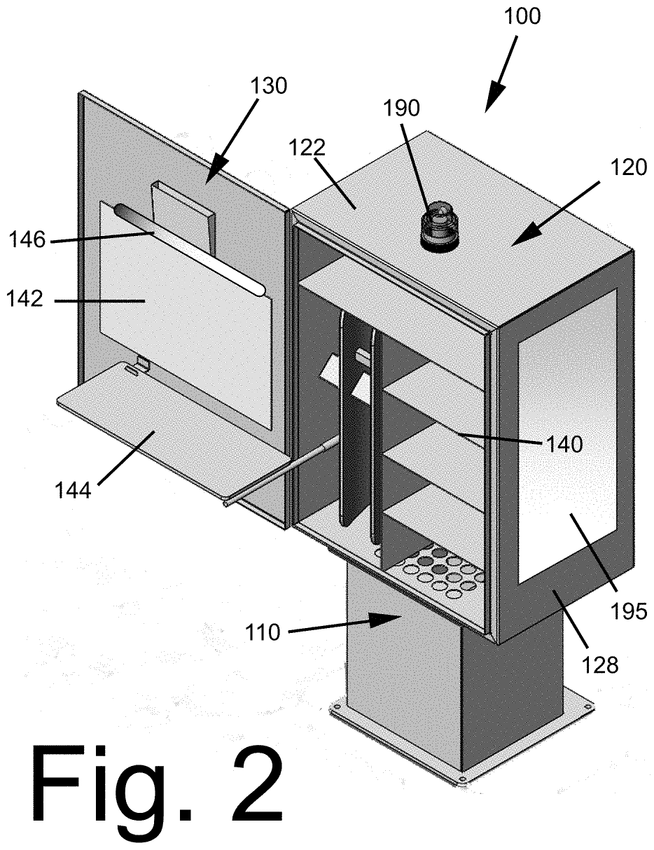

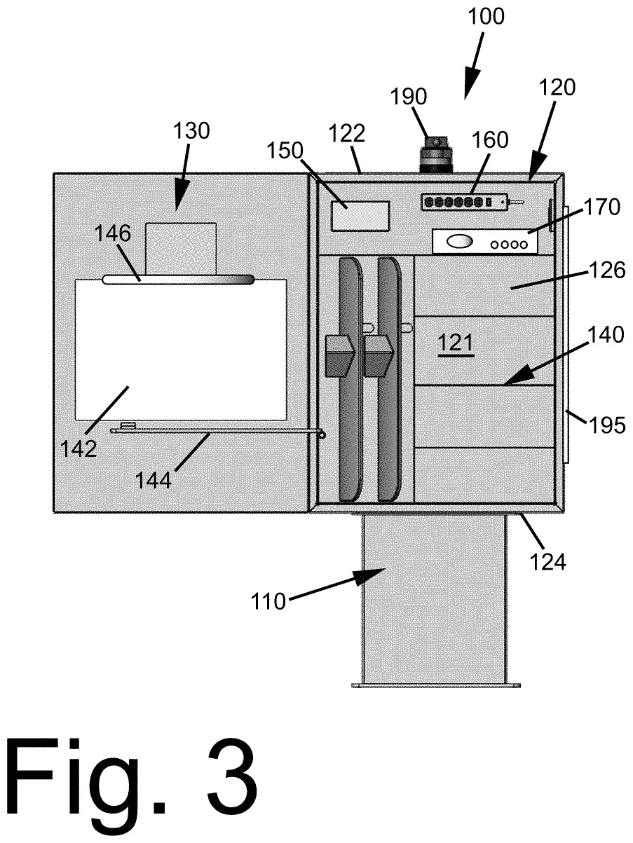

Referring now to the drawings, FIGS. 1-3 illustrate an exemplary embodiment of the emergency response kiosk, which is designated generally as reference numeral 100. Kiosks 100 are secure, stand-alone fixtures that store vital emergency response supplies and function as an on-site command and control center. Kiosks 100 are positioned at various strategic locations to provide first responders immediate decentralized access to vital medical supplies and equipment in the event of a mass casualty event. Each kiosk 100 also includes essential power, communication and tactical equipment for pre-staged command and control organization in the immediate location of any mass casualty event or critical incident. Each kiosk 100 includes an upright storage structure, which is suitable for both indoor and outdoor use. Kiosk 100 is designed to be tamper proof and to allow access only to authorized personnel.

Each kiosk 100 includes a raised container or locker box 120 mounted atop a pedestal base 110. Typically, pedestal base 110 and locker box 120 are fabricated from sheet metals or other suitable materials. Pedestal base 110 positions locker box 120 off the floor or ground at a height, which allows convenient access to its contents. Pedestal base 110 is typically bolted or otherwise secured to the ground or floor, preventing the kiosk from being easily toppled or moved.

Locker box 120 has a top wall 122, a bottom wall 124, a back wall 126 and two side walls 128, which define locker interior 121. Locker box 130 includes adjustable shelves and drawers 140, which can be selectively arranged to store and accommodate various supplies and equipment, such as medical slings-belts and packs, ballistic shields, small refrigeration units, automated external defibrillaters, and other medical and tactical equipment and supplies. The open front of locker box 120 is enclosed by a swinging locker door 130 that is hinged to one sidewall 128. Lock door 130 swings open to provide access to the locker interior 121. Locker door 140 also includes a large writing surface 142, a fold-out work desk, 144, a lamp 146, and various organizational pockets for storing documents, maps, plans, writing instruments and other administrative tools and supplies.

Kiosk 100 includes an access control mechanism 150. Access control mechanism 150 prevents unauthorized access to locker box 140, while ensuring convenient instant access to the locker box to first responders and other necessary personnel during emergencies. Access control mechanism 150 may incorporate any access and lock technologies for selectively permitting access to locker box 120, including conventional keyed and keypad locks, electronic and network enabled lock systems, radio-frequency fobs and remote lock systems, voice and bio-metric lock systems, and remote and network access systems. Access control mechanism 150 is selected for suitability to the particular kiosk application and location.

Kiosk 100 also hosts an array of electrical power sources and equipment. Kiosk 100 is typically connected to an external electrical power source (not shown), but may include its own backup power source, such as a generator or battery pack housed with pedestal base 110. An electrical power strip 160 is mounted to back wall 124 within locker interior 121, which can be used to power or charge various electrical devices. Kiosk 100 may include various radio and/or telephonic equipment 170 to coordinate command and control operations at the kiosk location. Kiosk 100 includes an internal video camera 180 for recording actions at the kiosk during emergencies. As shown, kiosk 100 includes a warning light 190, which typically activates when locker door 130 is opened. Kiosk 100 also includes a flat panel video display monitors 195 mounted to the exterior of back 126 and sidewalls 128. Monitors 195 are connected the kiosk's communication systems to display warnings, instructions and other information during emergency events. At other times, the monitors can be used for advertisement and other non-emergency messaging.

In certain other embodiments, kiosk may host exterior surveillance cameras for monitoring the the kiosk surroundings. In other embodiments, kiosk may incorporate an emergency “call box” fitted to the exterior of the locker box and integrated with the kiosk's hosted communication equipment. Other kiosk embodiments may include a self contained HVAC system for regulating the climate within locker interior 121. Typically, the HVAC system would be disposed within pedestal base 110.

It should be apparent from the foregoing that an invention having significant advantages has been provided. While the invention is shown in only a few of its forms, it is not just limited but is susceptible to various changes and modifications without departing from the spirit thereof. The embodiment of the present invention herein described and illustrated is not intended to be exhaustive or to limit the invention to the precise form disclosed. It is presented to explain the invention so that others skilled in the art might utilize its teachings. The embodiment of the present invention may be modified within the scope of the following claims.

I claim:

1. An emergency response kiosk for storing medical supplies, tactical gear and communication equipment, the kiosk comprising:
a pedestal base;
a locker box mounted atop the pedestal base, the locker box having an enclosure defining an interior thereof for receiving the stored medical supplies, tactical gear and communication equipment therein and an open front through which the interior is accessible;
a locker door hinged to the locker box to enclose the locker interior and pivot between an open position and a closed position; and
a latch mechanism for securing the locker door in the closed position and allowing selective access to the locker interior.

2. The kiosk of claim 1 wherein the locker box includes a shelf adjustably mounted within the locker interior.

3. The kiosk of claim 1 wherein the locker box includes a power source for powering the latch mechanism and the communication equipment.

4. The kiosk of claim 1 wherein the locker door includes a folding desk top.

5. The kiosk of claim 1 wherein the locker door includes a writing surface.

6. The kiosk of claim 1 wherein the locker door includes storage pockets.

7. The kiosk of claim 1 wherein the locker box includes a camera for monitoring the activities and action at the kiosk.

8. The kiosk of claim 1 wherein the locker box includes a warning light that activates when the locker door is in the open position.

9. The kiosk of claim 1 wherein the locker box includes video monitors.
